(19) United States
(12) Patent Application Publication
(10) Pub. No.: US 2026/0098828 A1
(43) Pub. Date: Apr. 9, 2026

(54) System for the simultaneous thermal analysis of a plurality of single samples of, in particular biological, material by means of differential scanning calorimetry (DSC), sample carrier and method for simultaneous analysis of a plurality of single samples

(71) Applicant: NETZSCH-Gerätebau GmbH, Selb (DE)

(72) Inventors: Rebekka Taubmann, Selb (DE); Florian Rummel, Selb (DE); Sabrina Küspert, Selb (DE); Markus Hollering, Wunsiedel (DE); Johannes Dunkel, Rehau (DE)

(21) Appl. No.: 19/351,659

(22) Filed: Oct. 7, 2025

(30) Foreign Application Priority Data
Oct. 9, 2024 (DE) 10 2024 129 206.7

(51) Int. Cl. G01N 25/48; G01N 33/483

(52) U.S. Cl. CPC G01N 25/4866; G01N 25/4813; G01N 25/4833; G01N 25/486; G01N 33/4833

(57) ABSTRACT

A system for the simultaneous thermal analysis of a plurality of single samples of, in particular biological, material by means of differential scanning calorimetry, with at least one sample carrier having several sample vessels, wherein a single sensor for measuring an amount of heat emitted or absorbed by the single sample during the thermal analysis is assigned to each sample vessel; a heating and/or cooling unit for the simultaneous temperature application of the single samples included in the sample vessels, with a receptacle for the at least one sample carrier; a measuring instrument, which is connected to the single sensors and which is formed to simultaneously capture a measuring value for the emitted or absorbed amount of heat of the single samples during the thermal analysis, a sample carrier, in particular for use in this system as well as a method for the simultaneous analysis of a plurality of single samples or groups of single samples by means of differential scanning calorimetry.

### TECHNICAL FIELD

The present invention relates to a system for the simultaneous thermal analysis of a plurality of single samples of, in particular biological, material by means of differential scanning calorimetry (DSC), a sample carrier, in particular for use in such a system as well as a method for the simultaneous analysis of a plurality of single samples.

### BACKGROUND

The differential scanning calorimetry (DSC) is a thermal analysis method for measuring an emitted or absorbed amount of heat of a sample during heat-up, cool-down or an isothermal process. The differential scanning calorimetry can be used for a plurality of analyses, such as, for example, the analysis of melting and glass transition temperatures, the degree of crystallization, the kinetic observation of chemical reactions, the specific heat capacity and of phase transitions. The differential scanning calorimetry (DSC) also permits using analyses for detecting diseases and in the medical field in research. Measuring equipment for performing analyses by means of differential scanning calorimetry typically permits only the simultaneous analyses of a few samples or single samples. In the case of a high sample throughput, in particular the waiting times for required analyses are thus very long and due to low capacities for analyses per time unit. Analyses can also not be performed on an economic scale because the single sample measurement is associated with a correspondingly high expenditure of time. Conventional measuring equipment for carrying out reproducible measurements also requires large sample volumes of 15 μm and more. Known measuring equipment thus does not provide for an economic use in the commercial sector.

Devices and methods are known as they are typically used for research purposes.

The US 2019/0003995 A1 describes a differential scanning calorimetry device for detecting diseases and monitoring the therapeutic efficacy by detecting heat-resistant variants of proteins and/or metabolic products in biological samples.

The WO 2017/066800 A1 describes a method for characterizing and/or predicting risks associated with a biological sample using thermal stability profiles.

### SUMMARY

It is the object of the present invention to create possibilities for the more cost-efficient, simpler and quicker performance of analyses by means of differential scanning calorimetry.

According to the invention, this object is in each case solved by the subject matters of the independent claims.

According to a first aspect of the invention, a system for the simultaneous thermal analysis of a plurality of single samples of in particular biological, material by means of differential scanning calorimetry (DSC) is provided. The system thereby comprises at least one sample carrier having several sample vessels, wherein a single sensor for measuring an amount of heat emitted or absorbed by the single sample during the thermal analysis is assigned to each sample vessel, a heating and/or cooling unit for the simultaneous temperature application of the single samples included in the sample vessels, with a receptacle for the at least one sample carrier, a measuring instrument, which is connected to the single sensors and which is formed to simultaneously capture a measuring value for the emitted or absorbed amount of heat of the single samples during the thermal analysis.

According to a second aspect of the invention, a sample carrier is provided, in particular for the use in the above-mentioned system, wherein the sample carrier has a plurality of sample vessels, which are preferably arranged in a defined grid, for a respective single sample.

According to a third aspect of the invention, a method for the simultaneous analysis of a plurality of single samples or groups of single samples of, in particular biological, material by means of differential scanning calorimetry (DSC), in particular in an above-mentioned system, is provided. The method thereby comprises the steps of: introducing the single samples into sample vessels in a sample carrier, in particular a sample carrier as described above, with single sensors assigned to the respective sample vessel, introducing the sample carrier into a heating and/or cooling unit, connecting the single sensors to a measuring instrument, performing a thermal analysis with simultaneous measurement of emitted or absorbed amount of heat of the single samples during the thermal analysis by means of the single sensors, simultaneously capturing measuring values of the single samples or groups of single samples by means of the measuring instrument, sending the measuring values to an evaluation unit, which communicates with the measuring instrument, and simultaneously evaluating the measuring values and deducing characterizing data structures of the single samples or groups of single samples on the basis of the measuring values by means of the evaluation unit.

According to a fourth aspect of the invention, the use of a system according to the invention and/or of a sample carrier according to the invention for the simultaneous thermal and/or visual analysis of in particular biological single samples by means of differential scanning calorimetry (DSC) is provided.

An idea on which the present invention is based is to carry out reproducible measurements even of smaller sample volumes with high sample throughput with simultaneously reduced expenditure of time for, for example, sample preparation and analysis of the measuring results. The invention can thereby accelerate the product development cycles in particular in, but not limited to, the biotechnological development as well as shorten waiting times for analysis results in particular in the medical diagnosis. The proposed invention also permits the simultaneous analysis of several samples under identical analysis conditions and the deduction of differentiated measuring and analysis results therefrom.

Advantageous embodiments and further developments follow from the subclaims, which are dependent on the independent claims, as well as from the description with reference to the figures.

According to an embodiment of the system, the receptacle is arranged in a test chamber, wherein the environmental conditions within the test chamber are defined or are formed in a variably adjustable manner. The simultaneous analysis of several samples can thus be performed in one measuring cycle and under the same environmental or measuring conditions, respectively, or by identical variation of the environmental conditions, respectively, for example in the course of an analysis. This offers advantages during the sample throughput but also with regard to the reproducibility of the measuring results and permits performing simultaneous analyses of several similar sample series under identical conditions. In this context, the defined or variably adjustable environmental conditions within the test chamber are in particular selected from: temperature, pressure, relative humidity, (inert) gas atmosphere and combinations thereof.

According to a further development, an evaluation unit is further provided in the system according to the invention, which evaluation unit is formed to receive the measuring values from the measuring instrument, to analyze them and to evaluate characterizing data structures on the basis of the measuring values for each single sample. The system thus permits the simultaneous deduction of data structures from measuring values of single samples, which were captured under identical conditions, and which are thus completely comparable with regard to all parameters. Achieving reproducibility across a large number of measurements and samples thus becomes possible and is improved.

According to a further development, a display is provided in the system, which is formed to visualize measuring values and/or data structures. The direct display of the course of the analysis and of the captured measuring values and/or data structures is thus significantly simplified and permits real-time monitoring of the analysis and of the captured measuring values prior to or after an evaluation and the direct display as well as the comparison of the measuring values and analysis results for the user. This also offers advantages under the aspects of quality monitoring and can contribute to the efficiency increase during the system utilization. The display can simultaneously be used to display the analysis parameters. If the display is formed, for example, as touchscreen, it simultaneously serves for the system control and for the input or selection, respectively, of analysis parameters or analysis programs of the system.

According to a further embodiment of the system, the single sensors are combined in a sensor plate. The number of the single sensors of the sensor plate thereby corresponds to the number of the sample receiving vessels in the sample carrier. The single sensors are thereby arranged at positions corresponding to positions of the sample vessels in the sensor plate. The use of a sensor plate contributes to the simplification of the system and to the improved positioning of the sensors, which are combined in the sensor plate, and which are fixed at defined, standardizable positions in the sensor plate, relative to the sample or to the sample vessel, respectively. A consistently high quality of the measuring results is also ensured thereby because the positioning precision and positional accuracy of the sensors is ensured.

In this context, a further advantageous embodiment of the system according to the invention provides that the sensor plate is made available as separate element, which can be connected in a positive or non-positive manner to the sample carrier. The sensor plate can thus be connected to the sample carrier in a simple manner, for example plugged into it, latched to it, or can be connected to the sample carrier by means of a clip connection or the like. The measurement or the measures preparing the measurement, respectively, such as, for example, sample preparation or preparation of the sample carriers, is significantly accelerated thereby and the positioning is improved and becomes reproducible. The data lines connected to the sensors can also be combined in the sensor plate and can be integrated into the system via a connection, for example plug connection.

According to a further alternative embodiment of the system, it is provided that the sensor plate is formed as element, which is permanently arranged in the receptacle, and remains there. To carry out the measurement, the sample carrier has to only be attached to, plugged into or latched or clipped, respectively, to the sensor plate, which is preferably fixed in a stationary manner in the receptacle. Due to the fact that the sensor plate permanently remains in the receptacle and thus in the system, the positioning of the sample carrier can be accomplished significantly more easily and more quickly. Due to the fact that the sensor plate does not directly come into contact with the sample, there is no risk of contamination or cross-contamination within the system. There is also no necessity to clean or to sterilize the sensor plate in a complex manner after the measurement, whereby the user-friendliness of the system can be increased and the sample throughput per time unit can be increased.

According to a further, alternative embodiment of the system according to the invention, a single sensor, which is integrated into each sample vessel or a single sensor, which can be connected in a positive or non-positive manner to each sample vessel, is provided. An individual control of each single sample vessel becomes possible hereby. This embodiment also makes it possible that only partly filled sample carriers can be evaluated.

According to a further embodiment, a surface area of the single sensor essentially corresponds to a surface area of the sample vessel. This ensures that measuring values can be captured across the entire surface area of the sample vessel and the measuring accuracy is thus increased significantly.

According to a further embodiment, the evaluation unit further comprises an interface or communication interface, respectively, which is formed to establish a communication connection between the evaluation unit and an external communication participant. This makes it possible that the evaluation unit transmits data sets from the system, in particular on data storages, such as a cloud or the like, or to servers without itself being communicatively coupled to a server or the like. The data can be transmitted more easily in this way and can be protected during the transmission, if necessary. The interface can thereby be formed for the wired or wireless data transmission. A transmission, for example via Bluetooth, a WLAN connection or with a different communication standard known to the person of skill in the art, is possible thereby and is captured by the invention.

According to a further development, the system further comprises a lighting unit, wherein the lighting unit is selected from: a UV light lighting unit, a lighting unit, which is designed to emit visible light, a lighting unit, which is designed to emit infrared light, a lighting unit, which is designed to emit polarized light, a lighting unit, which is designed to emit fluorescent light, in particular blue, green or red fluorescent light. The system thus provides for a simultaneous spectral analysis of the samples during the thermal analysis and expands the measuring and data range, which can be captured by means of the system. Processes within the sample can also be visually detected or made visible, respectively, during the thermal analysis.

According to a further embodiment of the system, the heating and/or cooling unit, the receptacle for the at least one sample carrier, the measuring instrument, the evaluation unit, the test chamber, the lighting unit and/or the display are at least partly surrounded by a housing, in particular a common housing. The system or at least components of the system are thus combined in a compact manner in a unit and the formation of a system, which is portable in a mobile manner, is also made possible.

According to a further embodiment of the system, an interface and/or a viewing window for the arrangement of an optical evaluation device, in particular a camera device, a microscope device, a fluorescence microscope or a Raman device is provided. A visual inspection of the course of the analysis method and an optical analysis of the samples is made possible hereby and the evaluation of the measuring results is thus improved or the bandwidth of the analysis methods, respectively, which can be performed with the system, is expanded, this in particular in connection and by using the above-described lighting unit and the lighting options provided thereby.

According to a further embodiment of the system, the interface and/or the viewing window are provided so as to be integrated into the housing and/or the test chamber. The integration of the interface provides for the simple and quick connection of the system to third-party devices and the connection of optical evaluation devices, while the viewing window provides direct optical access to the samples and permits the visual evaluation, for example by means of camera apparatus or microscope.

According to a further embodiment, the sample carrier is formed as microtiter plate with standardized configuration and the receptacle is formed as insert tray for the microtiter plate. A high sample throughput can be made possible in this way, while the microtiter plate, independently of its respective configuration, also permits the measurement of small volumes of in particular less than a sample volume of 10 μl in the single sample. The user-friendliness of the system is significantly improved by means of the insert tray because a reproducible positioning of the microtiter plate can be made in the system prior to each measurement.

According to a further development of the sample carrier, the latter is formed as a standardized microtiter plate made of a temperature-resistant material, in particular plastic material, with between 6 and 1546 sample receiving vessels. The simultaneous measurement of a plurality of samples is made possible with high reproducibility in this way, and the sample throughput is significantly increased thereby. The use of standardized microtiter plates, for example in the so-called 96-well microtiter plate format, which is typical in laboratories, additionally permits the likewise standardized embodiment of the above-described sensor plate for the connection thereto. A positionally exact arrangement of the measuring sensors relative to the sample vessels in the microtiter plate is simplified thereby. The sample preparation, which can also be performed in an automated manner due to the standard format of the sample carrier, is likewise accelerated significantly, with corresponding advantages for the sample throughput. The standardized microtiter plate can additionally be inserted into correspondingly adapted receptacles in the measuring system, and the system can thus be fitted quickly and in a simple manner with a plurality of samples. After completion of the analysis, the microtiter plate can be discharged or cleaned and reused, wherein the system is immediately available for the next measurement again. According to a further embodiment of the sample carrier, the plastic material has a temperature resistance in a temperature range of between −200°C. and +250° C, preferably of between −80° C. and +200° C. This permits a measurement in a high temperature range, without an impairment of the sample carrier taking place during the measurement. It is furthermore also possible that sample carriers coming directly from a low-temperature storage can be used for the measurement. A plurality of samples can thus be prepared for the measurement, can be stored at lowest temperatures, for example −80° C., and can then be processed in the system, without further preparatory measures being necessary prior to the respective analysis or measurement, respectively. The efficiency of the system and the sample throughput is increased thereby.

According to a further embodiment of the sample carrier, it is provided that a single sensor for measuring the amount of heat emitted or absorbed by the single samples during a thermal analysis is assigned to each sample vessel. The system thus operates with a plurality of single sensors, which simultaneously carry out the measurements. Due to the clear assignment of the single sensor to a sample vessel, it is ensured that only the respective single sample is measured by means of the sensor. The single sensors are sufficiently miniaturized thereby, so that measuring values from single samples in the above-described microtiter plate can be captured in a reliable and reproducible manner even in the smallest measuring volumes.

According to a further embodiment of the sample carrier, it is provided that the respective single sensor can be integrated into the sample vessel or can be connected in a positive or non-positive manner to the sample vessel. A sample carrier can be provided in this way, in the case of which a single sensor is in each case arranged in each or also only in single sample vessels. A sample carrier is thus provided, which is suitable for the measurement in the above-described system, and which is prepared accordingly. Said sample carrier can be provided as consumable article or as accessory for the system.

In an alternative embodiment, it is possible that the respective single sensor is connected to the respective sample vessel as needed. This permits the use of standardized sample vessels, which can be prepared for the measurement by fitting with single sensors. The sample vessels, for example standardized microtiter plate, can be separated from the single sensor, disposed of or cleaned and used again after the use. However, the flexibility of the system but also the efficiency when carrying out measurements and thus the sample throughput can also be increased hereby.

According to a further embodiment, it is provided that the single sensors are combined in a sensor plate and that the number of the single sensors of the sensor plate corresponds to the number of the sample receiving vessels in the sample carrier. The single sensors are thereby arranged at positions corresponding to positions of the sample vessels in the sensor plate. The use of a sensor plate significantly simplifies the handling of the system because the sensor plate, which in its surface area and arrangement of the sensors, corresponds to the configuration of the sample carrier and the arrangement of the sample vessels provided there, can be connected to the sample carrier prior to the measurement.

The connection can thereby take place, for example, by plugging into, latching to, clipping on or in another suitable manner, for the formation of a positive or non-positive connection. The connection between sensor plate and sample carrier thereby has a high positional accuracy, so that it is ensured that the samples included in the sample vessels are measured in a reproducible manner and with high accuracy. After the analysis has taken place, the sensor plate can be separated in a simple manner from the sample carrier and can be provided for the next measurement. A modular system is thus provided, which can also be used highly efficiently and for the processing of a high sample throughput, whereby time and cost savings can be realized.

According to an embodiment of the method, the analysis is performed under defined or variably adaptable environmental conditions. The environmental conditions are thereby selected from temperature, pressure, relative humidity, (inert) gas atmosphere and combinations thereof. It is thereby provided in the method that the environmental conditions are kept the same during the measurement by means of the system or that corresponding profiles for the environmental conditions, i.e., for example, a variable temperature, variable pressure, a variation of the humidity or of the gas atmosphere, are generated in the system, whereby the behavior of the samples can be measured under varying parameters of the environmental conditions. The respective environmental conditions in the system are monitored by means of suitable sensors, which are arranged, for example, in the test chamber or the housing as described above. The environmental conditions can thereby likewise be displayed on the above-described display or can be output so as to be integrated into the data sets, in order to ensure complete traceability and/or reproducibility of the measurement.

According to a further development of the method, the latter further comprises a visual analysis of the single samples or groups of single samples by means of an optical evaluation device, in particular a camera device, a microscope device, a fluorescence microscope or a Raman device. Additionally, or alternatively to the capture of the measuring values via the above-described sensors, a visual evaluation of the samples can thus also take place during the analysis process, which permits drawing additional conclusions about the composition of the sample or the behavior of the sampled materials during the thermal analysis. The visual inspection of the single samples or groups of single samples thereby preferably takes place via the above-described interface or a corresponding viewing window in the test chamber or the housing of the system. It is also possible that the capture of visual measuring values takes place in an automated manner in the system and that corresponding data sets are output by the system, combined with the data sets captured by the sensors. The combination of captured sensor values and additionally or alternatively captured visual parameters of the samples increases the flexibility and thus the field of application of the system, which can thus be adapted in a simple manner for different measuring tasks or can be provided for them.

It is provided in a further development of the method that the analysis is performed in a defined temperature profile. This can be performed in a simple manner as well as reproducibly and can be controlled automatically or manually via the above-described variation option of the environmental conditions.

In a preferred embodiment of the method according to the invention, it is provided that a simultaneous evaluating of the single samples is performed in an automated manner or manually in the evaluation unit. Due to the fact that a plurality of single samples is simultaneously analyzed thermally in the system according to the invention, the sample throughput can be increased significantly, and the efficiency of the system can be improved. A plurality of data sets can thus be provided, which permits drawing direct conclusions to the sample behavior or the composition of the samples, respectively, in response to automated evaluation. Comparisons between different samples are thus made possible in a single measuring or analysis pass. The option for the manual evaluation permits the user to adapt parameters during the analysis or for the subsequent analysis process, respectively. A corresponding software, which captures all measurements carried out in parallel and evaluates them simultaneously, is provided in the system for this purpose. The software can thereby be connected to an integrated database, so that completely automated evaluations are possible. The manual evaluation is also possible parallel to this or as alternative. Each sample can additionally or alternatively be analyzed optically during the measurement. The corresponding measuring values are thereby likewise captured in an automated manner or manually and are preferably evaluated in a software-supported manner.

In a further development, the method further comprises the step of visualizing the data structures, in particular on a display. The analysis can be tracked in real time in this way, wherein the captured measuring values are additionally transferred to a following evaluation entity via the above-described interface. Due to the visualization of the data structures on a display, measuring errors or malfunctions of the system can additionally be determined quickly.

Where this makes sense, the above embodiments and further developments can be combined as desired. Further possible embodiments, further developments and implementations of the invention also comprise combinations, which are not mentioned explicitly, of previously described features of the invention or of features, which will be described below with regard to the exemplary embodiments. The person of skill in the art will thereby in particular also add individual aspects as improvements or additions to the respective basic form of the present invention.

### BRIEF DESCRIPTIONS OF THE DRAWINGS

The present invention will be explained in more detail below on the basis of exemplary embodiments with reference to the enclosed figures of the drawings, in which:

FIG. 1 shows a schematic illustration of a sample carrier according to an exemplary embodiment of the invention in the top view;

FIG. 2 shows a schematic illustration of a further embodiment of the sample carrier according to a further exemplary embodiment of the invention in side view;

FIG. 3 shows a schematic illustration of a system according to an exemplary embodiment of the invention in perspective illustration; and

FIG. 4 shows a flow chart of a method for the analysis, in particular for the analysis by means of differential scanning calorimetry (DSC), of biological material, according to an exemplary embodiment of the invention.

### DETAILED DESCRIPTION

Unless stated otherwise, identical, functionally identical and identically acting elements, features and components are in each case provided with the same reference numerals in the figures of the drawing.

Even though specific embodiments and further developments are illustrated and described in the present case, the person of skill in the art will prefer that a plurality of alternative and/or similar embodiments can replace the illustrated and described specific exemplary embodiments, without turning away from the scope of the present invention. This application is to generally cover all modifications or changes of the specific exemplary embodiments described herein.

The enclosed figures are to convey a further understanding of embodiments of the invention and, in connection with the description, serve the purpose of explaining principles and concepts of the invention. Other exemplary embodiments and many of the mentioned advantages result with regard to the drawings. The drawings are to only be understood as schematic drawings and the elements of the drawings are not necessarily illustrated to scale. Terminology specifying a direction, such as, for instance, “top”, “bottom”, “left”, “right”, “above”, “below”, “horizontal”, “vertical”, “front”, “rear” and similar details are used only for explanatory purposes and do not serve the purpose of limiting the generality to specific designs as shown in the figures.

Dashed lines in the figures of the drawings clarify that the connections between the components connecting the dashed lines do not mandatorily have to have physical contact with one another but can likewise be coupled wirelessly with one another.

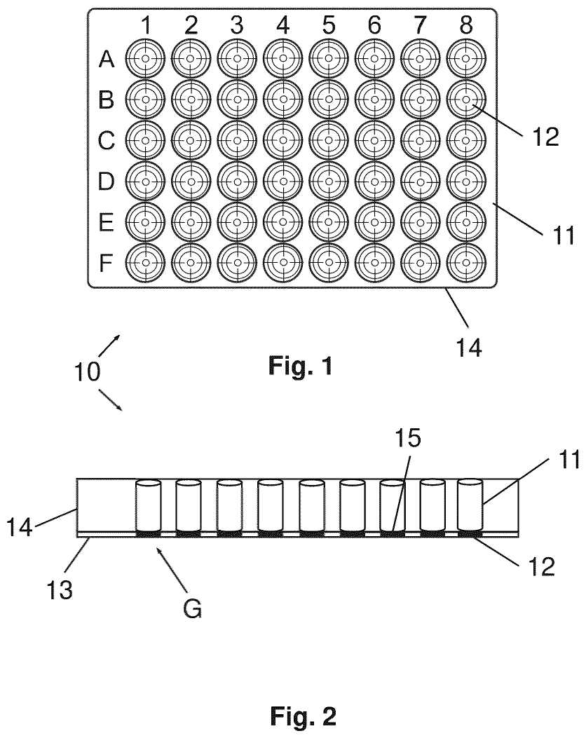

FIG. 1 shows a schematic illustration of a sample carrier 10 according to an exemplary embodiment of the invention in top view. The sample carrier 10 illustrated here is a microtiter plate 14 with a total of 56 single sample vessels 11, which are arranged in a standardized grid in the microtiter plate 14. It goes without saying that the sample carrier 10 is not fixed on the embodiments and configuration illustrated here. Microtiter plates 14 with fewer or more sample vessels 11 can likewise also be used in the system 20 according to the invention. The system 20 according to the invention, is designed for the use of sample carriers 10 with in particular between 6 and 1536 sample vessels 11 and permits a measurement, which is defined for each single sample, and which is reproducible. The sample carrier 10 according to the invention, permits the measurement of small volumes, i.e., of samples with a sample volume of 10 μl or less.

Standardized microtiter plates 14, which are available in the laboratory environment, can be used in the system 20 according to the invention. In the embodiment according to FIG. 1, the sample carrier 10 has single sensors 12, which are integrated into the respective sample vessels 11. The single sensors 12 measure the emitted or absorbed amount of heat of the single samples during the thermal analysis by means of differential scanning calorimetry (DSC). The embodiment illustrated in FIG. 1 shows an integrated sample carrier 10, i.e., the single sensors 12 and the sample carrier 10 or the sample vessels 11, respectively, are firmly connected to one another and the single sensors 12 are integrated into the sample vessels 11. In an alternative embodiment, which is not illustrated here, it is also possible that the single sensors 12, in the form of sensor plates, which have the same surface area as the sample vessels 11, are releasably connected individually to the respective sample vessels 11, for example plugged into them, clipped to them or are connected to them in another way. The use of single sensors 12 thus provides for the individual configuration of the sample carriers 10, i.e., as needed.

FIG. 2 shows a schematic illustration of a further embodiment of the sample carrier 10 according to a further exemplary embodiment of the invention in side view. The individual sensors 12, which have already been described in connection with FIG. 1, are combined here in a sensor plate 13, which is arranged below the microtiter plate 14. This sensor plate 13 has a number of single sensors 12, which corresponds to the number of the sample vessels 11 in the sample carrier 10 and which are joined firmly into a unit, the sensor plate 13. Prior to performing a thermal analysis by means of differential scanning calorimetry (DSC), this sensor plate 13 is connected to the sample carrier 10, in the exemplary embodiment a microtiter plate 14, and remains in this position during the measurement. The connection to the sample carrier 10 thereby takes place in a releasable manner, i.e., the sensor plate 13 is plugged into the sample carrier 10, is latched to it or clipped to it. The sensor plate 13 is thereby configured in such a way that a positionally accurate arrangement on the microtiter plate 14 is possible. After the sensor plate 13 is arranged on the sample carrier 10, a single sensor 12 is in each case located below each sample vessel 11 and covers the entire surface area G thereof. A complete measurement across the entire sample vessel 11 thus takes place. After completion of the analysis, the sensor plate 13 is removed from the sample carrier 10 and can be connected immediately to a further sample carrier 10 again, which is intended for the subsequent measurement. Due to the fact that the sensor plate 13 does not come into contact with the samples to be measured but a measurement takes place through the sample carrier 10 or the bottom 15 of the sample carrier 10, respectively, there is no risk of a contamination of the sensor plate 13, so that a cleaning prior to use can be omitted for the subsequent measurement. That being said, the sensor plate 13 can be made of a correspondingly sterilizable material, wherein the single sensors 12 are embedded in a liquid-and gas-tight manner in the sensor plate 13. With respect to the geometric dimension as well as the number of the single sensors 12 in the sensor plate 13, the latter can be adapted to different configurations of sample carriers 10, so that a matching sensor plate 13 is always provided for the respective sample carrier 10. The sample carrier 10 can be, for example, a microtiter plate 14, which can be used in the laboratory environment, and which has between 6 and 1536 sample vessels 11, so-called wells. The single sensors 12 are thereby adapted to the respective existing surface area of the single sample vessels 11. The single sensors 12 are sufficiently miniaturized thereby, in order to ensure a surface coverage of the sample vessels 11 or of the bottom 15 of the sample vessels 11, respectively, without being influenced in the measurement by adjacent sample vessels 11 thereby. The respective data lines (not illustrated) to the single sensors 12 are also combined in the sensor plate 13, so that the sensor plate 13 has a single interface, which is connected to the system 20, in order to export the captured measuring values from the system 20 and to provide it to an evaluation unit 16. For the thermal analysis, the sensor plate 13 is fastened sample carrier 10 and the total unit consisting of sample carrier 10 and sensor plate 13 is then inserted into the system 20. Alternatively, it is also possible that the sensor plate 13 is already installed in the analysis system and only the sample carrier 10 with the single samples, which are received therein, and which are to be measured, is inserted into a receptacle located there and is thereby or only subsequently connected to the sensor plate 13.

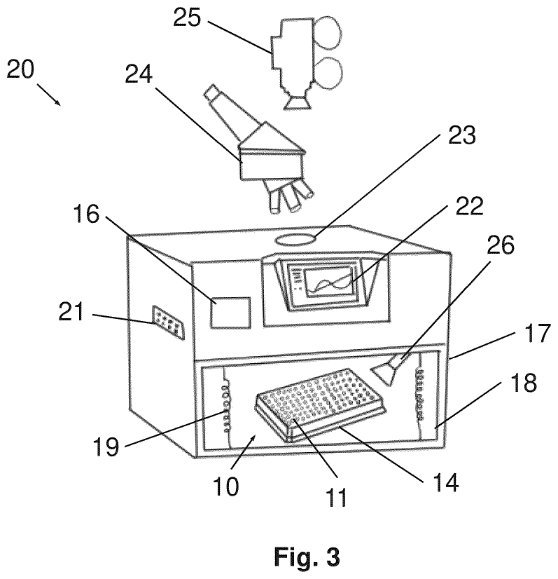

FIG. 3 shows a schematic illustration of a system 20 according to an exemplary embodiment of the invention in perspective illustration. The system 20 according to the invention, thereby comprises a housing 17, which includes a test chamber 18, into which the sample carrier 10, which is filled with the samples to be analyzed, is placed. Assigned to the test chamber 18, a heating or cooling unit 19, via which a temperature application of the samples can be carried out by means of defined temperature profiles, is located in the housing 17. A receptacle, which is not illustrated here, for the insertion of the sample carrier 10, is located in the test chamber 18. In the exemplary embodiment of FIG. 3, the sample carrier 10 is a correspondingly configured microtiter plate 14 with a plurality of sample vessels 11, which are firmly combined in the microtiter plate 14. These sample vessels 11 are filled with the respective samples prior to the thermal analysis and are then subjected to a simultaneous thermal analysis in the system according to the invention. For this purpose, a sensor plate 13 is located so as to be arranged below the sample carrier 10, as it has already been described in connection with FIG. 2. The single sensors 12 are assigned to the respective samples and capture temperature changes in the respective sample there during the thermal analysis. The sensor data is evaluated directly in the system 20. For this purpose, the system 20 comprises an evaluation unit 16, to which sensor data is transmitted and is provided for the evaluation. It is possible at the same time to export the analysis data to a downstream evaluation entity (not illustrated) via the interface 21, which is present in the system 20 and which is arranged on the housing 17. This can be, for example, a processing unit with corresponding evaluation software. Due to the embodiment of the system 20 illustrated in the exemplary embodiment, a direct evaluation of the captured values can also take place by means of the evaluation unit 16. The evaluated results are then visually displayed on the display 22, which is arranged in the housing 17. The display 22 also serves the purpose of displaying the operating parameters of the system 20, for example a temperature change, a temperature gradient or other environmental conditions, which are adjusted in the test chamber 18 and which belong to the parameters of the respective analysis method. The display 22 can also be formed as touchscreen and can be used as input means for controlling the system 20. Parameters can be changed, parameters can be input, or the measurement can be started or ended, respectively, via this display 22. In the exemplary embodiment, the display 22 is firmly connected to the housing 17, it goes without saying that it is also possible to provide a separate display 22, which is connected to the system 20 via the above-mentioned interface 21. It goes without saying that the display 22 can also be part of a processing unit, which is likewise integrated into the system 20 or which is formed to be capable of being connected to the system 20.

In the exemplary embodiment, the housing 17 additionally has a viewing window 23, via which a visual inspection of the samples is possible. The visual inspection can thereby take place by means of a microscope 24 or a camera apparatus 25, a fluorescence microscope or a Raman device, which are in each case connected to the system 20, i.e., arranged in the region of the housing 17 or on or in the housing 17. In the exemplary embodiment according to FIG. 3, a lighting unit 26 is located in the test chamber 18 itself, which lighting unit is formed as UV light lighting unit, lighting unit, which is designed to emit visible light, lighting unit, which is designed to emit infrared light, lighting unit, which is designed to emit polarized light, lighting unit, which is designed to emit fluorescent light, in particular blue, green or red fluorescent light. The visual inspection of the samples during the thermal analysis is supported by means of this lighting unit 26.

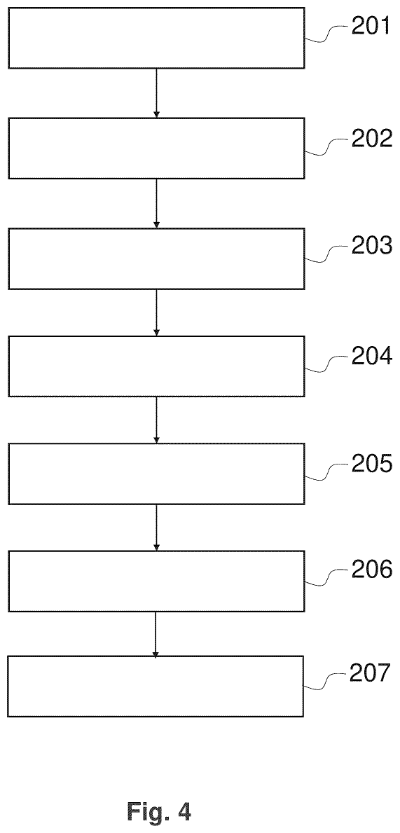

FIG. 4 shows a flowchart of a method for the simultaneous analysis of a plurality of single samples or groups of single samples of an in particular biological material by means of differential scanning calorimetry (DSC), in particular in a system according to the invention as described above. The method thereby comprises the steps of introducing 201 the single samples into sample vessels 11 in a sample carrier 10, in particular a sample carrier 10 as described above, with single sensors 12 assigned to the respective sample vessel 11; introducing 202 the sample carrier 10 into a heating and/or cooling unit 19; connecting 203 the single sensors 12 to a measuring instrument; performing 204 a thermal analysis with simultaneous measurement of emitted or absorbed amount of heat of the single samples during the thermal analysis by means of the single sensors 12; simultaneously capturing 205 measuring values of the single samples or groups of single samples by means of the measuring instrument; sending 206 the measuring values to an evaluation unit 16, which communicates with the measuring instrument; and simultaneously evaluating 207 the measuring values and deducing characterizing data structures of the single samples or groups of single samples on the basis of the measuring values by means of the evaluation unit 16. In the method according to the invention, an analysis can be performed by means of differential scanning calorimetry (DSC), in particular of biological material, for example blood, urine, sweat or skin tissue of animal or human origin. Other materials can furthermore also be analyzed in the method. The method is thus not limited to the use with biological material.

For introducing 201 the single sample, the latter is placed or filled into a sample vessel 11. The sample vessel 11 is thereby part of a sample carrier 10, which comprises a plurality of sample vessels 11. This sample carrier 10 can be, for example, a microtiter plate 14 with standardized configuration and surface, the filling or placement of the sample can take place, for example, by means of pipetting. The sample is thereby applied to the single sensors 12 assigned to the respective sample vessel 11. However, these single sensors are not in direct contact with the sample but are separated therefrom by means of the sample carrier 10. That being said, the sample carrier 10 is configured in such a way that a loss-free measurement is possible by means of the single sensors 12.

To introduce 202 the sample carrier 10 into a heating and/or cooling unit 19, a test chamber 18, which is available there, is opened, and the sample carrier 10 is then inserted into a receptacle provided in the test chamber 18. The connecting 203 of the single sensors 12 to the measuring instrument takes place after the inserting. The single sensors 12 can thereby in each case be connected individually to the measuring instrument, for example via a plug connection. Alternatively, it is also possible that the respective single sensors 12 are combined in a plug connection, which is then connected to a corresponding interface within the test chamber 18. In an alternative embodiment, the single sensors 12 are combined in a sensor plate 13, which additionally has the correspondingly combined lines of the single sensors 12 and which is equipped with a plug connector for connection to the measuring instrument.

To perform the thermal analysis 204, a temperature application of the sample carrier 10 takes place in the test chamber 18. The application can thereby take place by means of a defined temperature profile or at a constant temperature. The temperature range can thereby be between −200° C. and +250° C., preferably between −80° C. and +200° C. With regard to the material, the system 20 as well as the sample carrier 10 and the single sensors 12 or the sensor plate 13, respectively, are embodied in such a way that the respective upper as well as lower temperature ranges do not lead to an impairment of the measuring performance or durability, respectively, of the elements. During the temperature application, the single sensors 12 detect the amount of heat emitted or absorbed by the single samples during the thermal analysis and is transferred to an evaluation unit 16 as sensor value. A simultaneous capturing 205 of measuring values of the single samples or groups of single samples by means of the measuring instrument is thereby provided in the method. This means that a plurality of single samples can be processed in a single measuring cycle and that the method thus offers the possibility of significantly increasing the sample throughput. When using a microtiter plate 14 with, for example, 96 sample vessels, 96 single samples can thus be analyzed simultaneously, and the corresponding measuring data can be output accordingly. Compared to conventional methods with single measurement of the samples, this results in a significant capacity and time gain during the analysis. The configuration of the system 20 thereby ensures that reliable single values for the respective single samples are captured and are made available for the analysis. The simultaneous analysis also permits a single or groupwise evaluation of measuring data for single or groups of single samples. The sending of the measuring values 206 to an evaluation unit 16, which communicates with the measuring instrument, takes place via an interface 21, which is provided in the system 20. The sending can thereby take place in a wired as well as wireless manner, for example via a Bluetooth or WLAN connection. The captured measuring values are thereby transferred to a downstream evaluation entity, for example a computer, which is equipped with corresponding software, and is further analyzed there. A visual display of the captured and or evaluated measuring values can simultaneously also takes place on a display 22, which is provided in the system 20 and via which the operating parameters of the system 20, such as, for example, the temperature gradient in the test chamber 18, can also be displayed in addition to the measuring results. During the simultaneous evaluating 207 of the measuring values and the deduction of characterizing data structures of the single samples or groups of single samples on the basis of the measuring values, the raw data provided by the single sensors 12 are processed and are provided for a detailed evaluation of the performed analysis. The detailed evaluation can thereby be performed in the evaluation unit 16 or in a downstream evaluation entity in a software-supported manner or manually.

Different features for improving the stringency of the illustration have been combined in one or several examples in the preceding detailed description. It should be clear thereby, however, that the above description is only of an illustrative, but in no way of a limiting nature. It serves to cover all alternatives, modifications and equivalents of the different features and exemplary embodiments. Many other examples will be immediately and directly clear to the person of skill in the art based on his/her technical knowledge in consideration of the above description.

The exemplary embodiments were selected and described in order to be able to represent the principles on which the invention is based, and its application possibilities in practice in the best possible way. Experts can thus optimally modify and use the invention and its different exemplary embodiments with regard to the intended use. The terms “including” and “having” are used as neutral terminologies for the corresponding terms “comprising” in the claims as well as the description. A use of the terms “a”, “an”, “one” is to furthermore not generally rule out a plurality of features and components, which are described in this way.

1. A system for the simultaneous thermal analysis of a plurality of single samples of, in particular biological, material by means of differential scanning calorimetry, with:
at least one sample carrier having several sample vessels, wherein a single sensor for measuring an amount of heat emitted or absorbed by the single sample during the thermal analysis is assigned to each sample vessel;
a heating and/or cooling unit for the simultaneous temperature application of the single samples included in the sample vessels, with a receptacle for the at least one sample carrier;
a measuring instrument, which is connected to the single sensors and which is formed to simultaneously capture a measuring value for the emitted or absorbed amount of heat of the single samples during the thermal analysis.

2. The system according to claim 1, wherein the receptacle is arranged in a test chamber and wherein the environmental conditions within the test chamber are defined or are formed in a variably adjustable manner.

3. The system according to claim 2, wherein the defined or variably adjustable environmental conditions within the test chamber are selected from: temperature, pressure, relative humidity, (inert) gas atmosphere and combinations thereof.

4. The system according to claim 1, wherein an evaluation unit is further provided, which is formed to receive the measuring values from the measuring instrument, to analyze them and to evaluate characterizing data structures on the basis of the measuring values for each single sample.

5. The system according to claim 1, wherein a display is further provided, which is formed to visualize measuring values and/or data structures.

6. The system according to claim 1, wherein the single sensors are combined in a sensor plate and the number of the single sensors of the sensor plate corresponds to the number of the sample vessels in the sample carrier and wherein the single sensors are arranged at positions corresponding to positions of the sample vessels in the sensor plate.

7. The system according to claim 6, wherein the sensor plate is made available as separate element, which can be connected in a positive or non-positive manner to the sample carrier.

8. The system according to claim 6, wherein the sensor plate is formed as element, which is arranged in the receptacle.

9. The system according to claim 1, wherein a single sensor, which is integrated into each sample vessel or a single sensor, which can be connected in a positive or non-positive manner to each sample vessel, is provided.

10. The system according to claim 1, wherein a surface area of the single sensor essentially corresponds to a surface area of the sample vessel.

11. The system according to claim 1, wherein the evaluation unit further comprises an interface, which is formed to establish a communication connection between the evaluation unit and an external communication participant.

12. The system according to claim 11, wherein the external communication participant is formed as data processing apparatus and is communicatively coupled to the evaluation unit via the interface.

13. The system according to claim 1, further comprising a lighting unit, wherein the lighting unit is selected from: a UV light lighting unit, a lighting unit, which is designed to emit visible light, a lighting unit, which is designed to emit infrared light, a lighting unit, which is designed to emit polarized light, a lighting unit, which is designed to emit fluorescent light, in particular blue, green or red fluorescent light.

14. The system according to claim 1, wherein the heating and/or cooling unit, the receptacle for the at least one sample carrier, the measuring instrument, the evaluation unit, the test chamber, the lighting unit and/or the display are at least partly surrounded by a housing, in particular a common housing.

15. The system according to claim 1, wherein an interface and/or a viewing window for the arrangement of an optical evaluation device, in particular a camera device, a microscope device, a fluorescence microscope or a Raman device is provided.

16. The system according to claim 14, wherein the interface and/or the viewing window is provided so as to be integrated into the housing and/or the test chamber.

17. The system according to claim 1, wherein the sample carrier is formed as microtiter plate with standardized configuration and the receptacle is formed as insert tray for the microtiter plate.

18. A sample carrier, in particular for use in a system for the simultaneous thermal analysis of a plurality of single samples of, in particular biological, material by means of differential scanning calorimetry, with:
at least one sample carrier having several sample vessels, wherein a single sensor for measuring an amount of heat emitted or absorbed by the single sample during the thermal analysis is assigned to each sample vessel;
a heating and/or cooling unit for the simultaneous temperature application of the single samples included in the sample vessels, with a receptacle for the at least one sample carrier;
a measuring instrument, which is connected to the single sensors and which is formed to simultaneously capture a measuring value for the emitted or absorbed amount of heat of the single samples during the thermal analysis, wherein the sample carrier has a plurality of sample vessels, which are arranged in a defined grid, for a respective single sample.

19. The sample carrier according to claim 18, wherein the sample carrier is formed as a standardized microtiter plate made of a temperature-resistant material, in particular plastic material, with between 6 and 1536 sample vessels.

20. The sample carrier according to claim 19, wherein the plastic material has a temperature resistance in a temperature range of between −200° C. and +250° C., preferably of between −80° C. and +200° C.

21. The sample carrier according to claim 18, wherein a single sensor for measuring the amount of heat emitted or absorbed by the single samples during a thermal analysis is assigned to each sample vessel.

22. The sample carrier according to claim 21, wherein the single sensor is integrated into the sample vessel or can be connected in a positive or non-positive manner to the sample vessel.

23. The sample carrier according to claim 18, wherein a number of single sensors corresponding to the number of sample vessels are combined in a sensor plate, which can be connected in a positive or non-positive manner to the sample carrier, and the single sensors are arranged at positions corresponding to the positions of the sample vessels in the sensor plate.

24. A method for the simultaneous analysis of a plurality of single samples or groups of single samples of, in particular biological, material by means of differential scanning calorimetry, in particular in a system, with:
at least one sample carrier having several sample vessels, wherein a single sensor for measuring an amount of heat emitted or absorbed by the single sample during the thermal analysis is assigned to each sample vessel;
a heating and/or cooling unit for the simultaneous temperature application of the single samples included in the sample vessels, with a receptacle for the at least one sample carrier;
a measuring instrument, which is connected to the single sensors and which is formed to simultaneously capture a measuring value for the emitted or absorbed amount of heat of the single samples during the thermal analysis, the method comprising the steps of:
introducing the single samples into sample vessels in a sample carrier, in particular a sample carrier, wherein the sample carrier has a plurality of sample vessels, which are arranged in a defined grid, for a respective single sample, with single sensors assigned to the respective sample vessel;
introducing the sample carrier into a heating and/or cooling unit;
connecting the single sensors to a measuring instrument;
performing a thermal analysis with simultaneous measurement of emitted or absorbed amount of heat of the single samples during the thermal analysis by means of the single sensors;
simultaneously capturing measuring values of the single samples or groups of single samples by means of the measuring instrument;
sending the measuring values to an evaluation unit, which communicates with the measuring instrument; and
simultaneously evaluating the measuring values and deducing characterizing data structures of the single samples or groups of single samples on the basis of the measuring values by means of the evaluation unit.

25. The method according to claim 24, wherein the analysis is performed under defined or variably adaptable environmental conditions, wherein the defined or variably adaptable environmental conditions are selected from: temperature, pressure, relative humidity, (inert) gas atmosphere and combinations thereof.

26. The method according to claim 24, further comprising a visual analysis of the single samples or groups of single samples by means of an optical evaluation device, in particular a camera device, a microscope device, a fluorescence microscope or a Raman device.

27. The method according to claim 24, wherein the analysis is performed in a defined temperature profile.

28. The method according to claim 24, wherein the simultaneous evaluating is performed in an automated manner or manually in the evaluation unit.

29. The method according to claim 24, further comprising: visualizing the data structures, in particular on a display.

30. A method of using a system for the simultaneous thermal analysis of a plurality of single samples of, in particular biological, material by means of differential scanning calorimetry, wherein the system includes:
at least one sample carrier having several sample vessels, wherein a single sensor for measuring an amount of heat emitted or absorbed by the single sample during the thermal analysis is assigned to each sample vessel;
a heating and/or cooling unit for the simultaneous temperature application of the single samples included in the sample vessels, with a receptacle for the at least one sample carrier;
a measuring instrument, which is connected to the single sensors and which is formed to simultaneously capture a measuring value for the emitted or absorbed amount of heat of the single samples during the thermal analysis and/or of a sample carrier, and
wherein the sample carrier has a plurality of sample vessels, which are arranged in a defined grid, for a respective single sample for the simultaneous thermal and/or visual analysis of, in particular biological, single samples by means of differential scanning calorimetry.
